(19) United States
(12) Patent Application Publication
(10) Pub. No.: US 2023/0333118 A1
(43) Pub. Date: Oct. 19, 2023

(54) IMMUNOASSAY ANALYZER, IMMUNOASSAY KIT AND METHOD FOR DETECTING ANALYTE IN LIQUID SAMPLE

(71) Applicant: MOMM DIAGNOSTICS GMBH, Basel (CH)

(72) Inventors: Michael D. Laufer, Menlo Park, CA (US); Mathias Wipf, Riehen (CH); Oren Knopfmacher, San Francisco, CA (US); Meike Herget, Woodside, CA (US)

(21) Appl. No.: 17/753,884

(22) PCT Filed: Sep. 21, 2020
(86) PCT No.: PCT/EP2020/076218

§ 371 Date: Mar. 17, 2022

Related U.S. Application Data
(60) Provisional application No. 62/903,172, filed on Sep. 20, 2019.

(51) Int. Cl. G01N 33/543; G01N 33/68

(52) U.S. Cl. CPC G01N 33/6872; G01N 33/5438; G01N 33/54386; G01N 2333/904; G01N 2333/916

(57) ABSTRACT

A method for detecting at least one analyte in a liquid sample, the method comprising steps of a) binding the at least one analyte in the liquid sample with a capture reagent; b) binding a conjugate to the at least one analyte or the capture reagent; c) providing a substrate and converting the5 substrate into a product via a reaction with the conjugate, wherein the product is provided in a test area; and d) detecting an electric potential difference and/or a change in an electric current between at least one working electrode in the test area and at least one reference electrode in a reference area which is different from the test area.

### TECHNICAL FIELD

The present invention relates to biochemical testing, and in particular to testing which combines immunoassays, such as Enzyme-Linked Immuno-Sorbent Assays (ELISA) and Lateral Flow Assays (LFA) with an electrical readout. Specifically, the present invention relates to an immunoassay analyzer, an immunoassay kit including the immunoassay analyzer, and a method for detecting at least one analyte in a liquid sample using the immunoassay analyzer or the immunoassay kit.

### BACKGROUND

The analysis of a single or multiple parameters, such as the presence and concentration of an analyte in a fluid helps to diagnose or monitor the state of a condition, disease or health. There exists a need for a way to quantify analytes, such as ions, molecules, proteins, and pathogens with a high selectivity over a large concentration range in complex media, such as body fluids at the point and time of patient care.

In the field of medical technology and diagnostics, a large number of devices and methods for detecting an analyte in a body fluid are known. Today, accurate diagnostic tests performed at clinical laboratories are able to quantify one or more analytes over a large concentration range. However, such tests are invasive (require ml of blood), costly (require expensive infrastructure and trained personnel), and require long turnaround times (several hours to days), as the samples have to be shipped to the clinical laboratory. Point-of-care testing (POCT) addresses some of these issues, enabling rapid testing at the place and time of patient care with no or decentralized infrastructure, ease of use, robustness, and low cost.

A large number of POCT systems are based on the use of test elements in the form of test strips, in particular capillary test strips based on immunochromatographic assays. Such immunochromatographic assays, also known as LFA, are simple devices intended to detect the presence of a target analyte in a liquid sample without the need for specialized and costly equipment. They are mostly based on immunoassays using antigen-antibody interactions to locally enhance the concentration of the analyte. The introduction of a label causes a local chromatographic change, indicating the presence of the analyte. A microporous membrane (cellulose, paper, silk, polymer, etc.) is used for immobilizing a capture reagent (for example, antibody, engineered protein, DNA, RNA, an aptamer, etc.). When the analyte-containing sample is absorbed from one end of the membrane strip, the analyte is transported to the capture reagent by the capillary force through membrane pores. A binding reaction between the analyte and capture reagent occurs, and unbound molecules are subsequently separated by the medium flow. Such a test provides a quick analysis of analyte and convenience of one-step detection where the analysis is completed upon the sample application alone.

Besides their advantages, immunochromatographic assays have the following drawbacks: The sensitivity of the tests is limited, and most tests are only qualitative. More recent advances have included analytical readers to improve assay sensitivity, remove reliance on user visual acuity and provide quantitative results. Most analytical readers are based on optical methods (e.g. colorimetric, fluorescence), making them expensive. Moreover, these methods are limited in dynamic range (analyte concentration range) and the sensitivity is still too low for some applications where analytes occur at low concentrations or the signal is affected by the optical properties of the media (e.g. blood). Using enzymes as the tracer and the signal generator has improved the sensitivity of LFAs. However, the problems related to optical readout remain.

Meanwhile, electrical biosensors have been shown to have tremendous potential due to their superior sensitivity, wide dynamic range and simplicity. Common applications include pH sensors, Clark oxygen electrode and glucose meters. However, the application of electrical biosensors in medical diagnostics is still limited. A major drawback is the limited selectivity of potentiometric and amperometric systems. Using labels to circumvent this problem increases the complexity of a test.

WO 2018/218254 A1, as an example in which immunoassay is combined with an electrochemical sensing device, discloses a system employing a portable lateral flow assay configured to detect analytes captured by hydrogel particles equipped with affinity baits and an electrical data acquisition system including a two-prong electrode across the test line of the lateral flow assay.

### DESCRIPTION OF INVENTION

The present invention has been made in view of the aforementioned problems of the existing immunochromatographic assays.

The present invention describes, inter alia, an immunoassay analyzer which may be used as a new POCT platform. The immunoassay analyzer combines the advantage of low-cost, rapid, and easy-to-use immunochromatographic assays with the sensitivity of enzyme-linked immunoassays and the sensitivity and simplicity of electrical biosensors. The present invention addresses the needs in medical technology and diagnostics to overcome the current drawbacks of POCT.

Specifically, in a first aspect, the present invention is directed to an immunoassay analyzer for detecting at least one analyte in a liquid sample. The immunoassay analyzer comprises a transport matrix for transporting the liquid sample to at least one immobilization area along the transport matrix. The immunoassay analyzer further comprises at least one capture reagent immobilized in the at least one immobilization area, the capture reagent capable of binding with and thereby capturing the at least one analyte. Moreover, the immunoassay analyzer comprises at least one amperometric and/or potentiometric sensor element comprising at least one working electrode and at least one reference electrode, wherein the working electrode is configured to contact with the immobilization area(s) and the reference electrode is configured to contact with a portion of the transport matrix other than the immobilization area(s). Such analyzer may be referred to as an “eFlow” analyzer in the context of the present disclosure.

The immunoassay analyzer as described herein refers to a device that may be used in a process of detecting, and preferably determining an amount and/or concentration of, at least one analyte in a liquid sample in accordance with the present invention, said process being further described below as a further aspect of the present invention.

The liquid sample comprises, for example, a body fluid, a liquid food or other solutions, such as aqueous solutions. Preferably, the liquid sample comprises the body fluid such as blood, urine, saliva, sweat, tears, breast milk, sputum, ejaculate, etc. The liquid sample may consist of the body fluid, such as blood, urine, saliva, sweat, tears, breast milk, sputum, ejaculate, i.e. this body fluid may be applied directly (for example without dilution and/or processing). However, body fluids may also be diluted and or processed (e.g., homogenized) before application, for example when feces samples are to be examined.

The analyte as described herein refers to one or more of ions, molecules, proteins, pathogens, DNA and/or RNA contained in the liquid sample.

The transport matrix as described herein refers to a structure which allows the liquid sample to be transported from an area to another area thereof. Preferably, the transport matrix comprises a microporous membrane strip, more preferably the membrane strip comprising a polymer such as nitrocellulose or an inorganic material such as glass fibers. Other exemplary materials that may be used for the transport matrix are paper, silk, and polymeric materials (e.g., micro-fibrous polymeric materials, such as nonwovens). The liquid sample in this context is transported by the capillary force through the membrane pores. Alternatively, the transport matrix may comprise a liquid flow cell.

At least one immobilization area is provided along the transport matrix. An immobilization area as described herein is an area of the transport matrix in which the at least one analyte will be immobilized. Preferably, such an immobilization of the analyte is realized by immobilizing a capture reagent in the immobilization area, the capture reagent being capable of binding with and thereby capturing - thus immobilizing - the at least one analyte.

Preferably, the capture reagent comprises an antibody, an engineered protein, DNA, RNA or an aptamer.

The immunoassay analyzer according to the first aspect of the present invention further comprises a sensor element for amperometric and/or potentiometric measurement. The sensor element comprises at least one working electrode configured to contact with the at least one immobilization area and at least one reference electrode configured to contact with a portion of the transport matrix other than the at least one immobilization area. That is, the at least one reference electrode does not contact with the at least one immobilization area when the immunoassay analyzer is used to detect the analyte. Accordingly, the sensor element may be used to measure differential signals at different locations along the transport matrix.

Preferably, the immobilization area and the portion of the transport matrix other than the at least one immobilization area are spaced from each other in the direction in which the medium flows along the transport matrix. The immobilization area may be located upstream or downstream of the portion of the transport matrix other than the at least one immobilization area.

Preferably, the working electrode is an ion-selective electrode (ISE) such as a pH electrode or an oxidation-reduction potential (ORP) electrode. Preferably, the working electrode comprises a noble metal such as platinum, gold, silver, or a combination thereof, or a layer of metal oxides such as Al₂O₃, HfO₂, Ta₂O₅, or a combination thereof. More preferably, for pH measurement, the working electrode comprises a layer of titanium superimposed by a layer of Ta₂O₅.

Preferably, the reference electrode comprises a metal (e.g. Ti, Ag, Au, Pt, or a combination thereof) or a metal salt (e.g. Ag/AgCl). Preferably, the reference electrode does not comprise a metal oxide (such as Al₂O₃, HfO₂ or Ta₂O₅). As one example, the reference electrode may comprise a layer of titanium superposed by a layer of platinum.

Preferably, the immunoassay analyzer according to the first aspect of the present invention is configured to detect more than one analyte in the liquid sample. In this connection, the transport matrix comprises a first immobilization area and a second immobilization area. The immunoassay analyzer comprises a first capture reagent immobilized in the first immobilization area and capable of binding with and thereby capturing a first analyte in the liquid sample. The immunoassay analyzer further comprises a second capture reagent immobilized in the second immobilization area, the second capture reagent capable of binding with and thereby capturing a second analyte in the liquid sample. The sensor element comprises a first working electrode and a second working electrode configured to contact with the first immobilization area and the second immobilization area, respectively. The sensor element, as described above, comprises the reference electrode configured to contact the transport matrix in a manner that that the reference electrode does not contact with either the first immobilization area or the second immobilization area.

Preferably, the first analyte and the second analyte, respectively, comprise any of the aforementioned analytes, provided that they are different from each other. Preferably, the first capture reagent and the second capture reagent, respectively, comprise any of the aforementioned capture reagents.

Preferably, the first working electrode and the second working electrode, respectively, are any of the aforementioned ISEs. Preferably, the first working electrode and the second working electrode, respectively, comprise any of the materials and combination of materials mentioned above with respect to the working electrode.

Preferably, the second immobilization area is downstream of the first immobilization area or the second immobilization area and the first immobilization area are located, respectively, in isolated flow paths. For example, the transport matrix may be separated into two strips (e.g., two parallel strips, for example two parallel strips extending next to each other) wherein the first immobilization area is provided on a first strip and the second immobilization area is provided on a second strip.

Preferably, the sensor element further comprises a backing and/or support structure on which the working electrode(s) and the reference electrode(s) are provided. The backing and/or support structure may comprise any suitable material such as a material having a proper mechanical strength and/or inert to components of the liquid sample. For example, the backing and/or support structure may comprise quartz, polymers (e.g. vinyl, acrylic, polyester, polyvinyl chloride, polystyrene, silicone, polyolefin, epoxy), or cellulose.

Preferably, the sensor element is adhered, e.g. by an adhesive, to the transport matrix or brought into contact with the transport matrix by a mechanical pressure in a manner that the working electrode(s) is in contact with the immobilization area(s) and the reference electrode is in contact with the portion of the transport matrix other than the immobilization area(s).

Preferably, the immunoassay analyzer further comprises a read-out circuit. The read-out circuit is configured to determine an electric potential difference between the at least one working electrode and the reference electrode and/or a change in an electric current flowing through the working electrode and the reference electrode. In a configuration where the sensor element comprises more than one working electrode, e.g. a first and a second working electrode, preferably, the read-out circuit is configured to determine the electric potential difference between the first working electrode and the reference electrode and/or a change in an electric current flowing through the first working electrode and the reference electrode, and the read-out circuit is more preferably configured to further determine an electric potential difference between the second working electrode and the reference electrode and/or a change in an electric current flowing through the second working electrode and the reference electrode.

Preferably, the read-out circuit is configured to determine a respective amount and/or concentration of the at least one analyte based on the respective electric potential difference and/or the respective change in the electric current. To quantify the concentration of a specific analyte, the read-out circuit may be initially calibrated by measuring the electric potential difference and/or the respective change in the electric current generated by known analyte concentrations. This calibration information may be used to quantify the analyte from a single electric potential difference and/or the respective change in the electric current reading.

In a configuration where the transport matrix comprises a microporous membrane strip, preferably, the immunoassay analyzer further comprises a sample pad at a first end of the membrane strip for introducing the liquid sample to the transport matrix. Preferably, the immunoassay analyzer further comprises a reagent pad at the first end of the membrane strip and in fluid communication with the at least one immobilization area. Preferably, the reagent pad comprises a conjugate and/or a substrate as further described below.

Specifically, the immunoassay analyzer may further comprise a conjugate, which as mentioned above may be contained in the reagent pad or in the absence thereof may be provided in a different manner (e.g. in the liquid sample). Preferably, the conjugate comprises a binding molecule specific to the at least one analyte or to the at least one capture reagent. Preferably, the binding molecule is an antibody, an engineered protein, DNA, RNA or an aptamer.

Preferably, the conjugate further comprises an enzyme and/or a redox-active molecule such as ferrocyanide ions, methylene blue or resazurin.

Preferably, the enzyme comprises glucose oxidase, glucose-6-phosphate dehydrogenase, urease, alkaline phosphatase and/or horseradish peroxidase.

Preferably, the conjugate further comprises microspheres or colloidal metal which, for example, may be attached to the binding molecule so that more enzymes and/or more redox-active molecules may be linked to one binding molecule, thereby enhancing the signal resulting from the subsequent enzymatic reaction and/or redox reactions (further described below).

The immunoassay analyzer may further comprise a substrate, which as mentioned above may be contained in the reagent pad or in the absence thereof may be provided in a different manner (e.g. in the liquid sample). The substrate is convertible into a product via a reaction with the conjugate, preferably via an enzymatic reaction.

Preferably, the substrate comprises D-glucose, glucose 6-phosphate/NADP⁺; nitro-blue tetrazolium/5-bromo-4-chloro-3′-indolyl phosphate (NBT/BCIP); para-nitrophenyl phosphate (p-NPP); 3,3′,5,5′-Tetramethylbenzidine (TMB)/H₂O₂ and/or 2,2′-azino-bis(3-ethylbenzothiazoline-6-sulphonic acid) (ABTS)/H₂O₂.

Alternatively, the conjugate may comprise a molecule convertible into a product via a reaction (for example, an enzymatic reaction with an enzyme or a redox-active reaction with a redox-active molecule). In this case, the conjugate may comprise, for example, D-glucose, glucose 6-phosphate/NADP⁺; nitro-blue tetrazolium/5-bromo-4-chloro-3′-indolyl phosphate (NBT/BCIP); para-nitrophenyl phosphate (p-NPP); 3,3′,5,5′-Tetramethylbenzidine (TMB)/H₂O₂ and/or 2,2′-azino-bis(3-ethylbenzothiazoline-6-sulphonic acid) (ABTS)/H₂O₂. An enzyme and/or redox-active molecule may then be added to the transport matrix after binding the conjugate with the analyte and/or with the capture reagent. As noted above, such enzyme may be glucose oxidase, glucose-6-phosphate dehydrogenase, urease, alkaline phosphatase and/or horseradish peroxidase. It may be considered in this case that the enzyme or redox-active molecule is added as part of the substrate.

In any case, the substrate preferably comprises a substance capable of lowering the diffusibility of the product, the substance, for example, comprising saccharides or gels. Alternatively (or additionally), the substrate and/or the conjugate may be chosen such that the reaction between the substrate and the conjugate produces a precipitate, preferably an enzymatic reaction between the substrate and the conjugate producing the precipitate. Without wanting to be bound by theory, it is believed that this may help keeping the product at the position(s) where the reaction occurs, i.e. at the immobilization area, thereby increasing the lateral resolution along the transport matrix.

In a second aspect, the present invention is directed to an immunoassay kit comprising an immunoassay analyzer according to the first aspect of the present invention for detecting at least one analyte in a liquid sample, a conjugate comprising a binding molecule specific to the at least one analyte, and a substrate convertible into a product via a reaction with the conjugate. The conjugate and the substrate, respectively, may be the same as those described in the context of the first aspect of the present invention.

In a third aspect, the present invention is directed to another immunoassay analyzer for detecting an analyte in a liquid sample. The immunoassay analyzer comprises at least one reference well and at least one test well, the reference well being connected to each test well via a microfluidic channel. The immunoassay analyzer further comprises at least one reference electrode in the reference well and at least one working electrode in the test well. Such analyzer may be referred to as an “eELISA” in the context of the present disclosure.

The immunoassay analyzer according to the third aspect may further comprise at least one microwell plate comprising the at least one reference well and the at least one test well.

The microwell plate may have an essentially flat shape.

The microwell plate may comprise at least three, at least five, or at least nine wells.

The test well(s) and/or the reference well(s) of the microwell plate may have a volume of less than 200 microliters, preferably less than 100 microliters or less than 75 microliters.

The immunoassay analyzer according to the third aspect may include two or more test wells. In this case, at least one working electrode may be provided in each test well, such as at least one first working electrode in a first test well and at least one second working electrode in a second test well.

The immunoassay analyzer according to the third aspect may further comprise two or more working electrodes in a single test well (or in each test well). When the immunoassay analyzer comprises both a first working electrode and a second working electrode in one test well, the first working electrode and the second working electrode may be electrically separated and/or spaced from each other.

The immunoassay analyzer according to the third aspect of the present invention also refers to a device that may be used in a process of detecting, and preferably determining an amount and/or concentration of, at least one analyte in a liquid sample in accordance with the present invention, said process being further described below as a further aspect of the present invention. The analyte and the liquid sample may be the same as those described in the context of the first aspect of the present invention.

Preferably, the microwell plate is a plate with multiple test wells and at least one reference well formed therein, the test wells being used as small test spaces. The microwell plate may be manufactured from a variety of materials including, for example, polydimethylsiloxane, polystyrene, polypropylene, polycarbonate, cyclo-olefins, glass and/or quartz.

Preferably, the reference well is connected to each test well via a salt bridge in the microfluidic channel. Preferably, the salt bridge comprises an electrolyte and/or a gel.

Preferably, the first working electrode and/or the second working electrode mentioned above are any of the working electrodes described in the context of the first aspect of the present invention. Preferably, the first working electrode and/or the second working electrode are an ISE such as a pH electrode or an ORP electrode. More preferably, the first working electrode is a pH electrode and the second working electrode is an ORP electrode.

Preferably, the first working electrode comprises a layer of titanium superimposed by a layer of Ta₂O₅. Preferably, the second working electrode comprises a layer of titanium superposed by a layer of platinum.

Preferably, the reference electrode comprises any material of the reference electrode described in the context of the first aspect of the present invention. Preferably, the reference electrode comprises a metal (e.g. Ti, Ag, Au, Pt, or a combination thereof) or a metal salt (e.g. Ag/AgCl). Preferably, the reference electrode does not comprise a metal oxide (such as Al₂O₃, HfO₂ or Ta₂O₅).

Preferably, the immunoassay analyzer further comprises a capture reagent immobilized in each test well, the capture reagent capable of binding with and thereby capturing the analyte. Alternatively or additionally, the analyte may be immobilized in one or more of the test wells in a different manner. The analyte and the capture reagent, respectively, may be the same as those described in the context of the first aspect of the present invention.

Preferably, the immunoassay analyzer further comprises a conjugate and/or a substrate. The conjugate and the substrate, respectively, may be the same as those described in the context of the first aspect of the present invention.

Preferably, the immunoassay analyzer according to the third aspect of the present invention further comprises a read-out circuit for determining a first electric potential difference between the first working electrode and the reference electrode and/or a second electric potential difference between the second working electrode and the reference electrode. More preferably, the read-out circuit is configured to determine an amount and/or concentration of the analyte based on the respective electric potential differences.

In a fourth aspect, the present invention is directed to another immunoassay kit comprising an immunoassay analyzer according to the third aspect of the present invention for detecting at least one analyte in a liquid sample, a conjugate comprising a binding molecule specific to the at least one analyte, and a substrate convertible into a product via a reaction with the conjugate. The conjugate and the substrate, respectively, may be the same as those described in the context of the first aspect of the present invention.

In a fifth aspect, the present invention is directed to a microwell plate for use in the immunoassay analyzer according to the third aspect or for use in the kit according to the fourth aspect, as described above. The microwell plate comprises multiple test wells and at least one reference well, the reference well being connected to each test well via a microfluidic channel. The microwell plate may comprise a first working electrode in each test well and/or a reference electrode in the reference well. Furthermore, a second working electrode in each test well may be provided. The plate may further comprise contact pads for connecting the electrodes, preferably in a removable manner (e.g., by removably plugging the microwell plate into a corresponding electrical connector), to read out circuitry.

In a sixth aspect, the present invention is directed to a method for detecting at least one analyte in a liquid sample. The method comprises steps of a) binding the at least one analyte in the liquid sample with a capture reagent; b) binding a conjugate to the at least one analyte or the capture reagent; c) providing a substrate and converting the substrate into a product via a reaction with the conjugate, wherein the product is provided in a test area; and d) detecting an electric potential difference and/or a change in an electric current between at least one working electrode in the test area and at least one reference electrode in a reference area which is different from the test area.

The analyte, the liquid sample, the capture reagent, the conjugate, the substrate and the product may be the same as those described in the context of the first aspect of the present invention.

Preferably, the method according to the sixth aspect of the present invention further comprises determining an amount and/or concentration of the at least one analyte based on the electric potential difference and/or the change in electric current. Preferably, this determination is made based on the electric potential difference and/or the change in an electric current detected in step d).

The method may be carried out using the immunoassay analyzer of either the first aspect or the third aspect of the present invention.

According to a first implementation of the method, the test area in which the product is provided is at least one immobilization area of a transport matrix of an immunoassay analyzer. The transport matrix may be the same as described above for the first aspect of the invention.

In this context, preferably, step a) comprises introducing the liquid sample to the transport matrix, the transport matrix comprising the test area (i.e. the immobilization area), wherein the capture reagent is immobilized in the test area. The analyte in the liquid sample is bound to the capture reagent and therefore also immobilized in the test area.

Preferably, step b) comprises providing the conjugate in the test area of the transport matrix. Preferably, providing the conjugate comprises steps of b1) providing the conjugate in the test area after the at least one analyte has bound to the capture reagent or b2) mixing the conjugate with the liquid sample before the liquid sample is introduced to the transport matrix. Preferably, step b2) further comprises mixing the liquid sample with a buffer solution containing the conjugate before the liquid sample is introduced to the transport matrix or providing the liquid sample in a reagent pad in contact and/or fluidly coupled with the transport matrix, wherein the reagent pad contains the conjugate.

Preferably, step c) comprises providing the substrate in the test area of the transport matrix, wherein providing the substrate comprises one of steps of c1) providing the liquid sample in a reagent pad or receptacle in contact and/or fluidly coupled with the transport matrix, wherein the reagent pad or receptable contains the substrate, c2) providing the substrate in the test area along with the liquid sample, and c3) providing a buffer solution containing the substrate in the test area after the liquid sample has been transported to the test area and the conjugate has been bound to the at least one analyte or the capture reagent.

Preferably, step d) comprises d1) providing a sensor element comprising the working electrode and the reference electrode and d2) contacting the sensor element with the transport matrix in a manner that the working electrode is in contact with the test area and the reference electrode is in contact with the reference area. Preferably, the reference area is a portion of the transport matrix other than the test area and/or a portion of the transport matrix in which the capture reagent is not immobilized. In particular, the reference area may be a portion of the transport matrix that is positioned upstream or, preferably, positioned downstream of the test area along the transport matrix.

The sensor element in this first implementation of the method may be the same as that described in the context of the first aspect of the present invention.

Preferably, this first implementation of the method is carried out using the immunoassay analyzer according to the first aspect of the present invention, as described above.

According to a second implementation of the method, the test area in which the product is provided is at least one test well.

Specifically, the method according to this second implementation preferably comprises providing at least one microwell plate comprising at least one reference well and at least one test well defining the test area. In this context, step a) preferably comprises providing the liquid sample in the test well and/or in the reference well. The microwell plate may be the same as that described in the context of the third aspect of the present invention.

The reference electrode may then be provided in the reference well, and the working electrode may be provided in the test well. The reference well is connected to the test well via at least one microfluidic channel. Preferably, step d) is carried out using the microwell plate.

Preferably, the method further comprises a step of immobilizing the capture reagent in the test well. Preferably, the method further comprises a step of immobilizing the analyte in the test well. Preferably, step b) comprises providing the conjugate in the test well and/or in the reference well. Preferably, step c) comprises providing the substrate in the test well and/or in the reference well.

Preferably, this second implementation of the method is carried out using the immunoassay analyzer according to the third aspect of the present invention, as described above.

Accordingly, the present invention is directed to an immunoassay analyzer and a method for determining the presence, and preferably the concentration, of at least one analyte in a liquid sample. The present invention is based on electrical readout of an immunoassay. In the present invention, the liquid sample containing the analyte is in direct contact with a transducer (i.e. a sensor element) for electrical detection of the analyte and omitting the use of expensive optical parts. Besides the low cost, the scalability of this approach has distinguishing advantages over conventional optical-based and electrochemical methods. The present invention allows to measure signals in microliter volumes and to test small sample volumes (a few µl). Further, multiplexing allows multi-parameter analysis in a single test with a small form factor.

The sensor element is integrated into a rapid immunosorbent assay, where the analyte may be first recognized and captured by an immobilized capture reagent, and second, sandwiched by a conjugate containing a secondary detection reagent linked to an enzyme (e.g. microspheres coated with antibodies and enzymes). Finally, the enzymatic conversion of a substrate is detected electrically via potentiometry or amperometry (e.g. with potentiometric pH and ORP measurements).

The combination of rapid assays, such as ELISA or LFA, with electrochemical readout (i.e. potentiometric or amperometric) allows a fast and quantitative detection of at least one analyte over a large concentration range (ranging from mg/ml down to pg/ml) in small sample volumes.

The general aspects of the present invention being described above, specific, non-limiting embodiments further illustrating certain details of the present invention will be described below with reference to the respective drawings, in which:

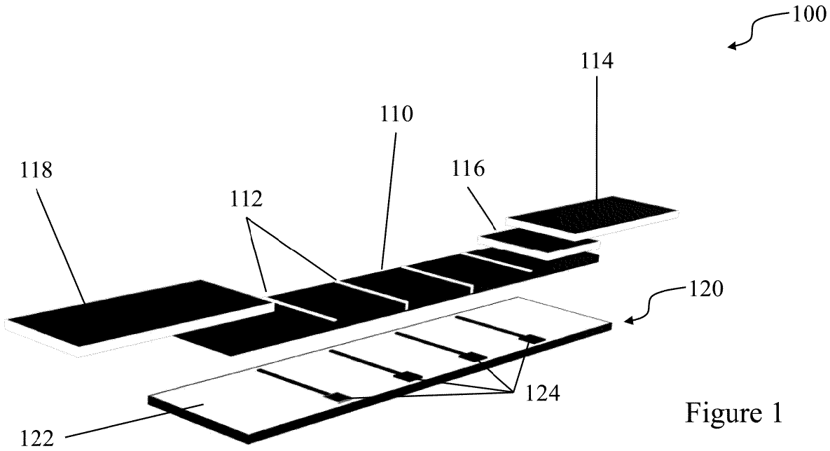

FIG. 1 shows a schematic of an eFlow test cartridge according to the first aspect of the present invention;

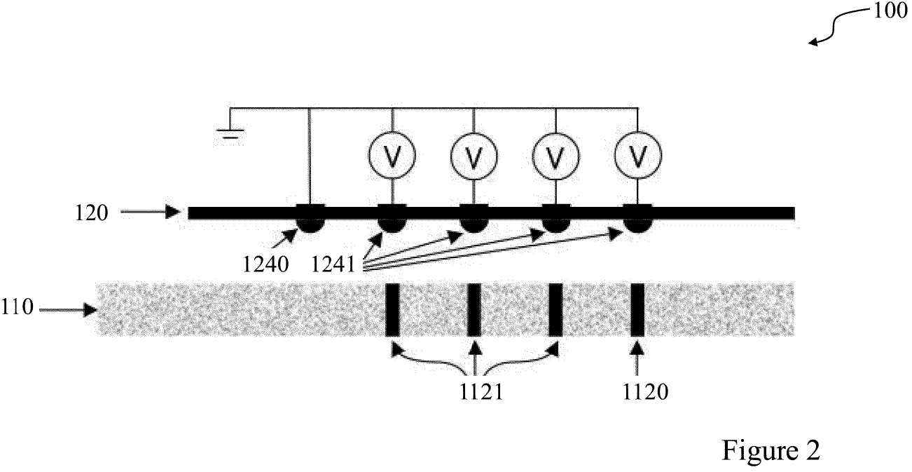

FIG. 2 shows a schematic side view of the eFlow test cartridge;

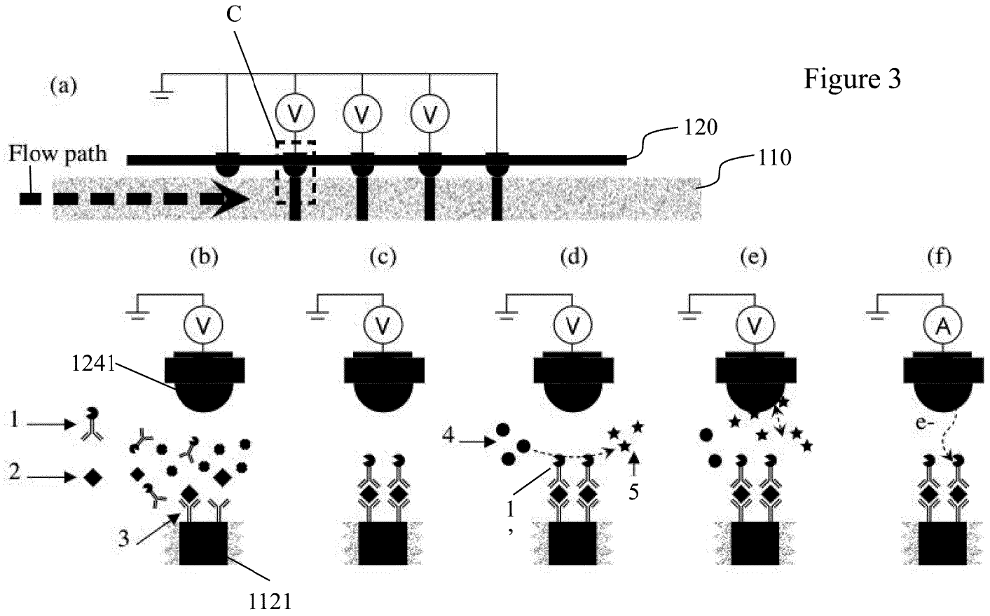

FIG. 3a shows a schematic side view of the eFlow test cartridge in which a sensor element is in contact with a transport matrix;

FIGS. 3b to 3f each show a detail of a specific part of the eFlow test cartridge;

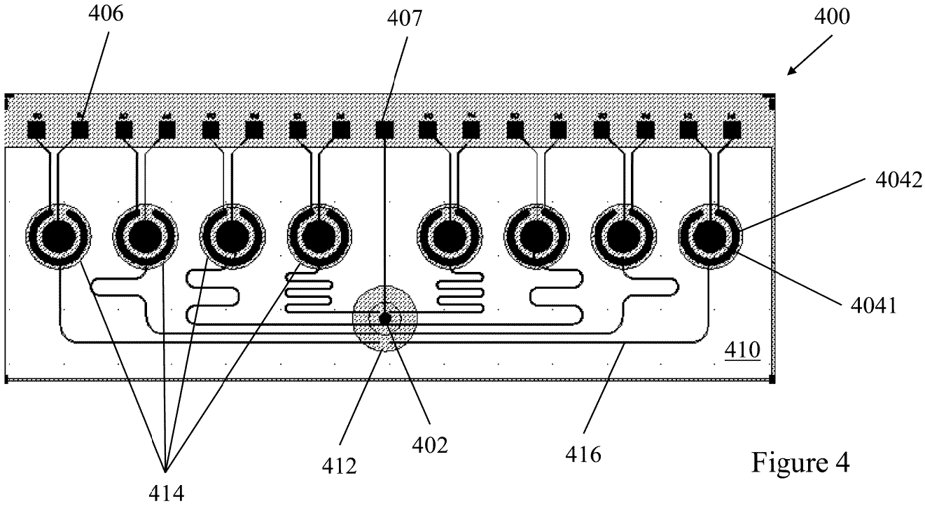

FIG. 4 shows a schematic of an eELISA sensor chip according to the third aspect of the present invention;

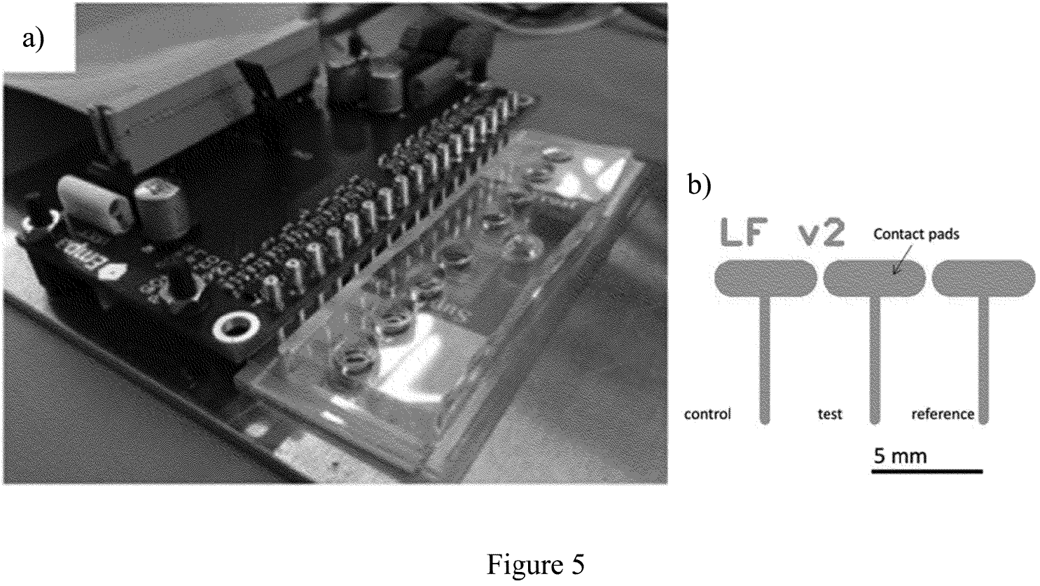

FIG. 5a is a picture of an eELISA sensor chip connected to a multi-channel read-out electronic board;

FIG. 5b shows a schematic of the eFlow sensor electrodes;

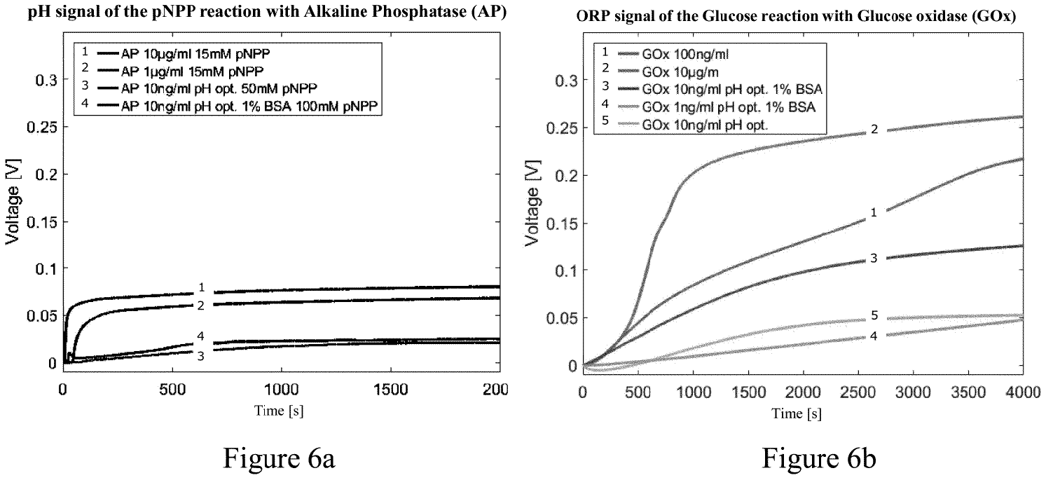

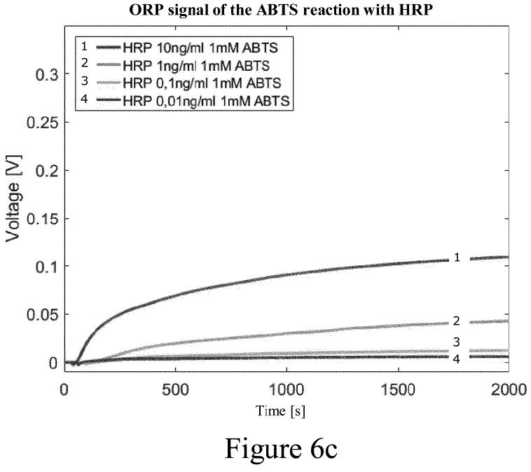

FIGS. 6a to 6c, respectively, show pH and ORP signal generation by different enzymes at various concentrations in the presence of their respective substrates;

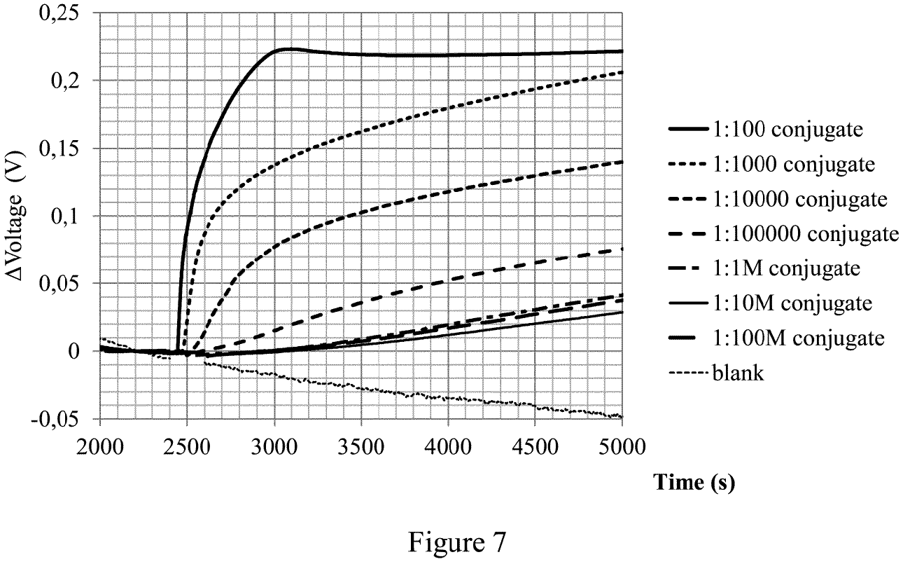

FIG. 7 shows the measurement results using an eELISA sensor chip;

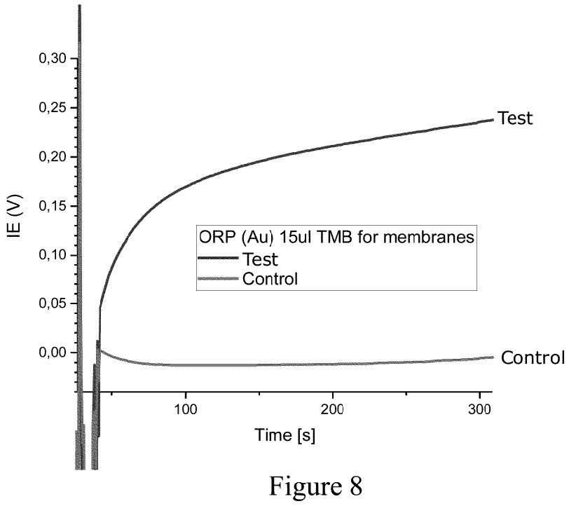

FIG. 8 shows the measurement results using an eFlow analyzer;

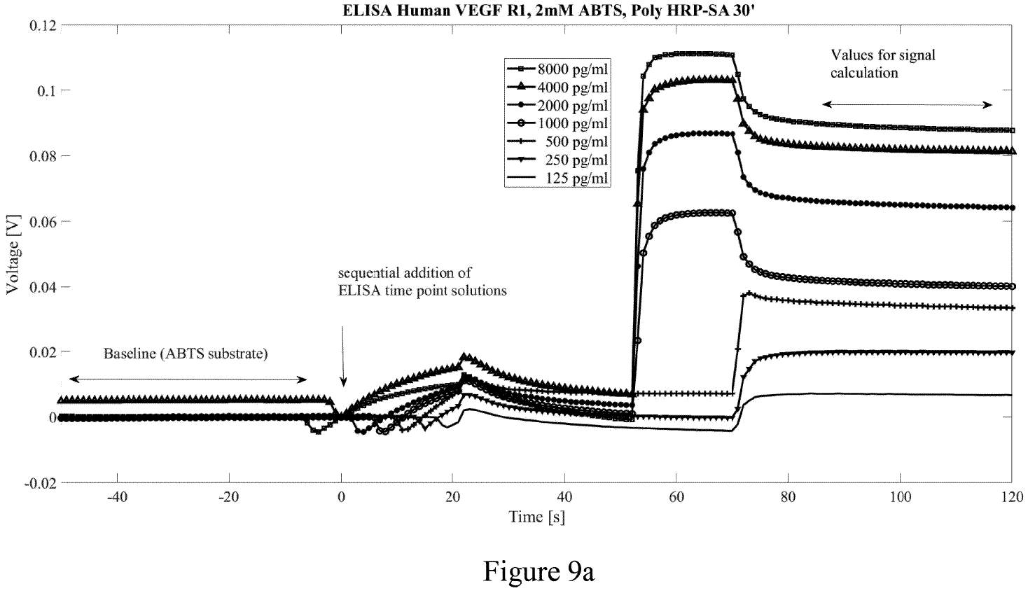

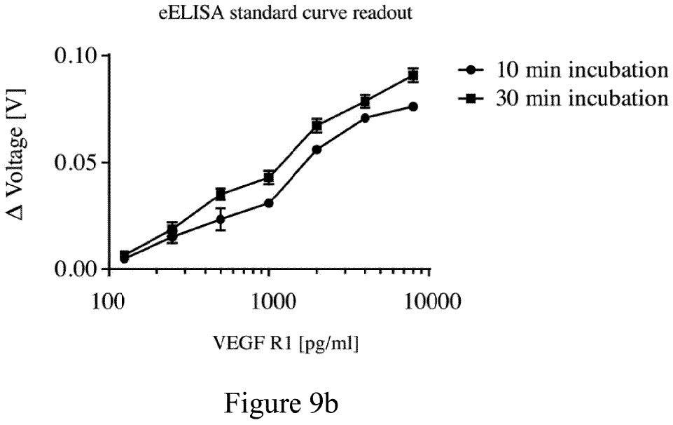

FIG. 9a shows further measurement results using an eELISA sensor chip;

FIG. 9b shows a standard eELISA curve derived from FIG. 9a;

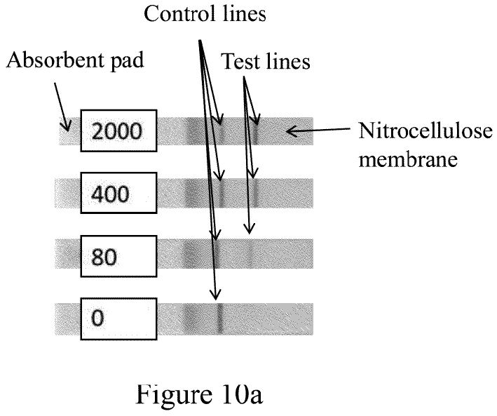

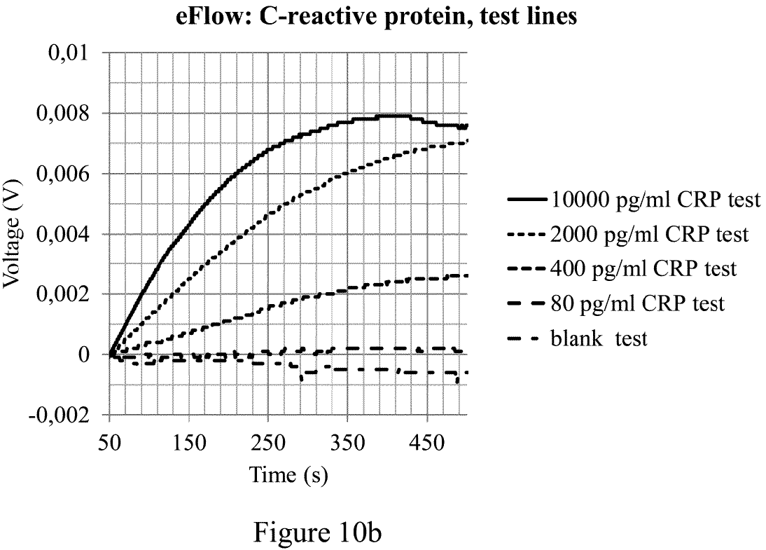

FIG. 10a shows various LFA test strips through which liquid samples having different protein concentrations are flowed; and

FIG. 10b shows further measurement results using an eFlow sensor chip.

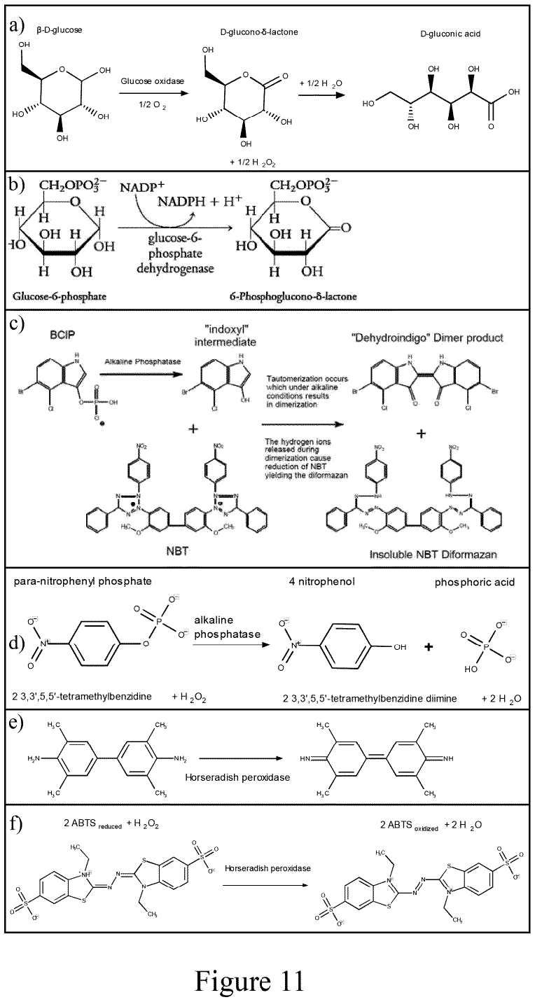

FIG. 11 illustrates exemplary reactions.

### eflow Analyzer

Referring to FIGS. 1 and 2, in accordance with the first aspect of the present invention, an immunoassay analyzer 100, also referred to as eFlow analyzer (or eFlow test cartridge) herein, comprises a transport matrix 110 for transporting a liquid sample to at least one immobilization area 112 along the transport matrix 110. The transport matrix 110 shown here is a membrane strip. Capture reagents specific to an analyte of interest are immobilized in at least one of the immobilization areas 112 (i.e. in each test immobilization area 1121, see FIG. 2).

The immunoassay analyzer 100 further comprises a sample pad 114 attached to a first end of the transport matrix 110 for introducing the liquid sample. At an opposing, second end in the longitudinal direction of the transport matrix 110 an absorbent pad 118 is attached. The sample pad 114 and the absorbent pad 118 define therebetween a flow path for the liquid sample, that is, the liquid sample, when introduced to the transport matrix 110, would flow sequentially through the respective immobilization areas 112 from the first end to the second end of the transport matrix 110 (see also FIG. 3a). The immunoassay analyzer 100 further comprises a reagent pad 116 attached at the first end of the transport matrix 110, between the sample pad 114 and the transport matrix 110, and in fluid communication with the respective immobilization areas 112 and also with the sample pad 114. Accordingly, components contained in the reagent pad 116 may be combined with components in the liquid sample when the liquid sample passes through the reagent pad 116 before flowing into the transport matrix 110.

The immunoassay analyzer 100 further comprises a sensor element 120. The sensor element 120 comprises a backing 122 and a plurality of electrodes 124 on the backing 122. Referring to FIG. 2, the sensor element 120 comprises a minimum of two electrodes 124, i.e., a reference electrode 1240 to set the potential of the liquid sample in the transport matrix and at least one working electrode 1241 to measure the voltage or current generated with respect to the reference electrode 1240 at an area of the transport matrix 110 different from that of the reference electrode. In the exemplary embodiment shown in FIGS. 1 and 2, a plurality of working electrodes 1241 is provided (e.g., four, as shown).

Still referring to FIG. 2, the immobilization areas 112 comprise the test immobilization areas 1121 in which the capture reagents for analytes are immobilized and a control immobilization area 1120 in which capture reagents with high affinity to conjugates (further described below) are immobilized. The control immobilization area 1120 is used for confirming the proper execution of the test. The working electrodes 1241 are configured to contact with a respective one of the immobilization areas 1120 and 1121 and the reference electrode 1240 is configured to contact with a portion of the transport matrix 110 other than the immobilization areas 1120 and 1121. This area may be referred to as the “reference area”. Preferably, the reference area is configured to not capture analytes and/or conjugates present in a liquid sample.

As the immunoassay analyzer 100 is used for detecting an analyte in a liquid sample, the liquid sample is introduced to the transport matrix 110, thereby binding the analyte with the capture reagent in the test immobilization areas 1121. Thereafter, a conjugate is provided to the transport matrix 110 and bound to the analyte at the respective test immobilization areas 1121. This may also be referred to as the analyte being sandwiched between the capture reagent and the conjugate in the context of the present disclosure. The skilled reader will appreciate that the analyte may, but does not have to, bind to the capture reagent and/or the conjugate directly. Also an indirect binding is possible. For example, one or more intermediate linking complexes, such as a primary and a secondary antibody, may be used.

Then, a substrate is provided to the transport matrix 110. The substrate is converted into a product via a reaction with the conjugate at the respective test immobilization areas 1121. Finally, an electric potential difference and/or a change in an electric current between a working electrode 1241 and the reference electrode 1240 is detected.

The above procedures will now be described in further details with reference to FIGS. 3a to 3F below.

FIG. 1a shows the sensor element 120 in contact with the transport matrix 110. The contact is maintained, for example, by an external pressure or an adhesive. The sensor element 120 may be oriented in different ways (i.e. contact the transport matrix 110 from the top, bottom, or side). The liquid sample is transported along the transport matrix 110 (i.e. along the flow path). As will be appreciated, the reference electrode is located upstream of the working electrodes along the transport matrix in this case, but this arrangement could also be reversed.

A dashed rectangle C in FIG. 3a indicates the detail shown in FIGS. 3b to 3f, each showing a more detailed schematic representation of a test immobilization area 1121 and a working electrode 1241.

Referring to FIG. 3b, as the liquid sample containing analytes 2 of interest is brought in contact with the transport matrix 110, the analytes 2 interact with, i.e. are captured by, the specific capture reagents 3 immobilized in the corresponding test immobilization area 1121. Conjugates 1 are introduced with or after the liquid sample. As shown in FIG. 3c, the conjugates 1 bind to the analytes 2 and are immobilized on the capture reagent 3. Unbound substances in the liquid sample are separated by the medium flow.

Referring to FIGS. 3d and 3e, the conjugate 1 contains an enzyme 1′. When a substrate 4 is introduced with or after the liquid sample, immobilized enzyme 1′ (as shown in an exemplary manner here, on conjugates 1) converts the substrate 4 into a product 5. The product 5 reacts with the working electrode 1121 arranged in proximity to the test immobilization area 1121 and generates a change in voltage, which may be detected using the sensor element 120.

Alternatively, referring to FIG. 3f, the conjugate 1 may contain a redox-active molecule, which exchanges electrons with the working electrode 1241 and generates a current, which may be detected using the sensor element 120.

It is noted that the processes shown in FIGS. 3b to 3f are based on a “sandwich” mechanism in which the analyte 2 is sandwiched by the capture reagent 3 and the conjugate 1. The present invention, however, is not limited thereto. The detection of the analyte may be carried out on the basis of a “competitive” mechanism in which a conjugate specific to the capture reagent (instead of being specific to the analyte) is provided, the analytes and the conjugates thereby competing for the capture reagents. In this case, the less analyte in the liquid sample, the more conjugate is retained in the test immobilization area and the stronger the signal.

More specifically, it should be noted that variations of the above procedure are possible in the context of the present invention. For example, in such competitive procedure both the conjugate and the analyte are configured to bind (directly or indirectly) to the capture reagent in a competitive manner. In such competitive procedure, the liquid sample is introduced to the transport matrix 110, thereby binding the analyte with the capture reagent in the test immobilization areas 1121. Thereafter, a conjugate is provided to the transport matrix 110 and bound to the capture reagent where no analyte has been bound. In other words, such competitive procedure may involve binding the conjugate not with the analyte.

The immunoassay analyzer 100 uses immunoassay techniques, such as ELISA, LFA tests, and improves the quantitative readout by enhancing the sensitivity and dynamic range of the test. This is enabled by electrochemical transducers (i.e. the sensor element 120 which includes the electrodes 124) in contact with the transport matrix 110. Compared to other proposed methods for electrochemical readout of immunochromatographic assays, the immunoassay analyzer 100 has the following distinguishable characteristics.

The use of two or more electrodes configured to be placed at different sections of the transport matrix 110 allows to measure differential signals (i.e. voltage difference between the electrodes, or a difference in current) proportional to the difference in local analyte concentration. Differential signals of electrodes at different locations along the transport matrix 110 allow to omit a reference (grounded) electrode or reference signal, quantify multiple analytes in a single test, and integrate calibration standards if needed.

The immunoassay analyzer 100 comprises the sensor element 120 in contact with the transport matrix 110 capable of transporting a liquid sample. The transport matrix 110 may contain one or several types of immobilized capture reagents, each with a high affinity for a specific analyte. This allows the simultaneous analysis of one or several different analytes.

The transport matrix 110 may comprise either
a) a microporous membrane strip (membrane). The membrane may be made from a polymer, such as nitrocellulose, or inorganic material, such as glass fiber. It is used for immobilizing capture reagents in specific test immobilization areas. When the analyte-containing liquid sample is absorbed from one end of the membrane strip, the analyte may be transported to the capture reagent by the capillary force through membrane pores. A binding reaction between the analyte and capture reagent may occur, and unbound molecules may subsequently be separated by the medium flow, or
b) a liquid flow cell containing immobilized capture reagents in a test immobilization area. The analyte-containing medium may flow through the flow cell and transport the analyte to the capture reagent. A binding reaction between the analyte and capture reagent may occur, and unbound molecules may subsequently be separated by the medium flow.

The conjugate comprises a binding molecule specific to an analyte of interest (for example, antibody, engineered protein, DNA, RNA, an aptamer, etc.) and either
a) enzymes that generate a detectable signal by enzymatic conversion of a substrate (for example: glucose oxidase, urease, horseradish peroxidase, alkaline phosphatase),
b) redox-active molecules (for example ferrocyanide ions, methylene blue, resazurin, etc.), or
c) a combination of the above.

The conjugate may further comprise microspheres or colloidal metal attached to the binding molecule so as to provide a greater surface area and/or more sites for connecting the enzymes and/or redox-active molecules.

The conjugate may be applied to the transport matrix 110 after the analyte has bound to the capture reagent. Alternatively, the conjugate may be mixed with the liquid sample before it is applied to the transport matrix 110, for example, by either
a) mixing the liquid sample with a solution (e.g., a buffer solution) containing the conjugate prior to application to the transport matrix 110, or
b) applying the liquid sample to the reagent pad 116 (e.g. by introducing the liquid sample to the sample sad 114 and allowing the liquid sample to flow into the reagent pad 116 and then into the transport matrix 110), the reagent pad 116 being in contact with the transport matrix 110 and containing the conjugate.

The analytes may bind to the conjugates and may be immobilized on the capture reagent or vice versa. The presence of analytes causes a local increase in conjugate concentration in the corresponding test immobilization area 112. The final concentration of immobilized conjugate across the transport matrix 110 depends on the analyte concentration and the location of the capture reagent. The conjugate then causes a local conversion of a substrate, generating, for example, either
a) a local chromatographic change,
b) a local pH change,
c) a local oxidation-reduction potential change, or
d) a local redox reaction, which indicates the presence of the analyte.

The amperometric or potentiometric sensor element 120 contains two or more electrodes 124, of which one may be used as the reference electrode 1240. The material at the surface of the electrode which is to be in contact with the liquid sample may contain a noble metal (for example, platinum, gold or silver), e.g. to measure faradaic currents, or oxidation-reduction potentials (ORP), or may contain a layer of metal oxides, such as Al₂O₃, HfO₂, Ta₂O₅, e.g. to measure a pH value. In another example, the electrode can be a combination of different layers of the same or different material. In case a membrane is used as the transport matrix 110, the contact of the electrodes to the membrane may be achieved by mechanical pressure or direct adhesion to the membrane from any side (top, bottom or side).

In case the conjugate contains an enzyme, the substrate which can be converted to the product by the enzyme may be, for example, either
a) added to the transport matrix on the reagent pad 116 by drying (i.e. lyophilization) and released upon application of liquid sample,
b) applied to the transport matrix 110 along with the liquid sample, or
c) applied to the transport matrix 110 subsequent to application of the analyte and the conjugate by applying a substrate buffer containing the substrate.

Depending on the enzyme/substrate combination, the conversion of the substrate by the conjugate may result in a local change in pH or ORP. Those changes induce either a voltage change on the adjacent sensor surface (potentiometric measurement) or a change in current (amperometric measurement). The changes (rate and amplitude) in voltage or current directly correlate with the amount of the analyte present. Analyte quantification may be done by measuring the difference in signal between different electrodes (e.g. differential signal between the electrodes close to the test immobilization areas 1121 and blank area in Fehler! Verweisquelle konnte nicht gefunden werden.). To increase the lateral resolution along the transport matrix, the substrate may contain a substance which lowers the diffusion rate of the product when applied (e.g. saccharides or gels), or the enzymatic reaction produces a precipitate. Alternatively, the transport matrix may be separated into parallel strips to avoid product diffusion between the test immobilization areas.

In case the conjugate comprises a redox-active molecule, a voltage or current is generated proportional to the analyte concentration in the proximity of the working electrode 1241 via electron exchange with the working electrode 1241. In another example, the interaction of the conjugate with the target analyte releases a third molecule of H⁺ or e⁻ which can be detected by the working electrodes (indirect detection).

The use of multiple electrodes at different test immobilization areas 1121 along the transport matrix 110 allows the individual measurement of local conjugate concentrations. As this corresponds to the local immobilization of the respective analyte, simultaneous quantification of multiple analytes is possible. A parameter analyzer such as a read-out circuit (a practical example described below) can be used to analyze the signal. The differential signal (voltage or current) between individual electrodes indicates the difference in conjugate and analyte concentration at the electrode locations. The relative concentration of two or more analytes can be extracted by analyzing the ratios of the signals at the corresponding immobilization areas. The absolute analyte quantification can be determined using the differential signal between the working electrodes at the test immobilization areas and an electrode (i.e. the reference electrode) which is preferably placed outside of an immobilization area where no conjugate or a known concentration of conjugate exists.

The eFlow analyzer as described above is a simple device intended to detect and quantify the presence of one or multiple target analytes in a liquid sample without the need for specialized and costly equipment like a spectrophotometer or optical lenses. The present invention provides a rapid analysis of analyte and convenience of one-step detection.

### eELISA Analyzer

Referring to FIG. 4, in accordance with the third aspect of the present invention, an immunoassay analyzer 400, also referred to as eELISA sensor chip (or eELISA system) herein, comprises a microwell plate 410. The microwell plate 410 comprises a reference well 412 and a plurality of (in FIG. 4, eight) test wells 414. The reference well 412 is connected to each test well 414 via a microfluidic channel 416. The immunoassay analyzer 400 further comprises a reference electrode 402 in the reference well 412. The immunoassay analyzer 400 further comprises, in each test well 414, a first working electrode 4041 and a second working electrode 4042. The first working electrode 4041 and the second working electrode 4042 are separated from each other.

The first working electrode 4041 is a pH electrode. The second working electrode 4042 is an ORP electrode. The first working electrode 4041 and the second working electrode 4042 are each connected to a contact pad 406 arranged to facilitate the electrical measurement. The reference electrode 402 is also connected to a contact pad 407. Accordingly, a potential difference between the reference electrode 402 and any of the first working electrodes 4041 and the second working electrodes 4042 may be determined.

The use of the eELISA sensor chip is further described in the following.

### EXAMPLES

The present invention is directed to quantitative readout of rapid tests, such as immunochromatographic and fluorescence-based assays, for the quantification of analytes, such as ions, small molecules, proteins, DNA, RNA and pathogens. The present invention can be implemented to detect an analyte of interest in environment, food, humans or animals.

The aforementioned eFlow analyzer may be developed for POCT, wherein the eFlow analyzer comprises single-use cartridges and a reader instrument. The test is developed in order to diagnose the state of a specific condition, disease or health by analyzing the ratio or concentration of analytes in a bodily fluid. The cartridge comprises the immunoassays for the target analytes (a transport matrix with immobilized capture reagents) and a sensor element. The cartridge will fit the reader instrument, which analyzes the electrode signals over time and communicate the test results via a graphical interface, or a wireless or cable communication.

In the following, the materials and methods used to demonstrate the concept of the eFlow analyzer are further described. Also, the eELISA system used to quantify analytes in bulk solution (microwell) is further described.

The eFlow analyzer combines potentiometric sensors with an enzymatically amplified lateral flow assay. Using the eFlow analyzer, the inventors demonstrate the rapid detection of C-reactive protein in a sample. Using the eELISA sensor chip, the inventors demonstrate the use of potentiometric sensors to monitor several enzymatic reactions generating a pH and ORP change and to quantify a specific biomarker (Human VEGF R1) in a sample.

### eELISA Sensor Chip Fabrication

An eELISA sensor chip contained eight 50 µl test wells and one 50 µl reference well. The latter is connected to each test well via a microfluidic channel. Each test well contained a pH and an ORP sensor electrode, the reference well contained a platinum reference electrode (see Fehler! Verweisquelle konnte nicht gefunden werden.a for the layout of such an eELISA sensor chip). The sensor and reference electrodes were fabricated by photolithography on a 4-inch quartz wafer (fused silica JGS2 wafer 4-inch, thickness = 500 ± 25 µm, 2-side polished, TTV < 10 µm - 1 SEMI Flat, MicroChemicals, Ulm, Germany). For the pH electrodes, 50 nm titanium was deposited by electron beam evaporation, followed by 20 nm Ta₂O₅ by atomic layer deposition

(ALD plasma Oxford). For the ORP and reference electrodes, 10 nm Titanium was deposited by electron beam evaporation, followed by 50 nm Platinum deposition by electron beam evaporation. The microwells and microfluidic channels were produced by pouring polydimethylsiloxane (PDMS, SYLGARD 184 Silicone Elastomer) onto SU-8 (SU-8 2000 MicroChem) patterned Si wafers, degassing, and heating at 60° C. for 2 h. Through holes for the microwells were punched used a 6 mm biopsy punch (Harris Uni-Core). The PDMS and sensor chips were cleaned in acetone and isopropanol, and 10 min UV-ozone. After cleaning, the PDMS was immediately pressed onto the sensor chip and heated to 120° C. for 10 min. The reference electrode system (microwell and the microfluidic channels) was filled with a reference gel made from 1 M KCl and 1% Agarose (Agarose II, VWR International, LLC) in deionized water. The gel was heated to 90° C. and 50 µl were pipetted into the reference well. Applying overpressure at the reference well, the microfluidic channels were filled up to the edge of the test wells. The gel was jellified at room temperature for 1 h.

### Multi-Channel Read-Out Electronics

The inventors designed a 16-channel readout circuit with electronic buffers to translate the very high output impedance of the pH and ORP sensor electrodes to a low output impedance. The interface to the sensor chips was done using spring-loaded contacts (16 low input current buffer amplifiers for connecting the sensor electrodes and 1 output for the reference electrode). The circuit was realized on the top and first inner copper layer of a four-copper layer board using 16 operational amplifier LTC6268 (Analog Devices, Inc.). The board was realized on a standard process using FR4 (Beta Layout GmbH, Aarbergen, Germany). The buffer amplifier output voltage was measured using a USB-6210 Multifunction I/O device (National Instruments).

For example, FIG. 5a shows a picture of an eELISA sensor chip connected to the multi-channel read-out electronic board.

### eFlow Sensor Chip Fabrication

An eFlow sensor chip contained 3 redox sensitive electrodes (ORP sensors). The electrodes were prepared by stencil lithography. Shadow masks (Layout see Fehler! Verweisquelle konnte nicht gefunden werden.c) were manufactured by Beta Layout GmbH, Aarbergen, Germany. Platinum or gold electrodes were deposited on a 4-inch quartz wafer (Fused silica JGS2 wafer 4 inch, thickness = 500 ± 25 µm, 2-side polished, TTV < 10 µm - 1 SEMI Flat, MicroChemicals, Ulm, Germany) using the stencil mask. 5 nm chromium adhesion layer and 50 nm platinum or 50 nm gold were evaporated using electron beam evaporation. After wafer dicing, the individual sensor chips were cleaned in acetone, isopropanol and 10 min UV-ozone.

For example, FIG. 5b shows a schematic of the eFlow sensor chip electrodes, including three electrodes with contact pads respectively for probing the control line, probing the test line, and defining the reference potential (from left to right).

### Enzymatic Signal Generation

The signal generated at the pH and ORP electrodes may be induced by an enzymatic reaction changing the pH or the oxidation-reduction potential of the media. The electrical potential generated at the electrodes can be described by the Nernst equation. Different enzyme-substrate systems, such as a) to f) summarized in Table 1, may be used for signal generation, the reactions being further illustrated below.

TABLE 1

| | | | |
|---|---|---|---|
| | Substrate | Product | Enzymes |
| a) | D-Glucose | Gluconic acid | Glucose oxidase |
| b) | Glucose 6-phosphate + NADP⁺ | 6-phospho-D-glucono-1,5- lactone + NADPH + H⁺ | Glucose-6-phosphate dehydrogenase |
| c) | nitro-blue tetrazolium/5- bromo-4-chloro-3′-indolyl phosphate (NBT/BCIP) | 5,5′-dibromo-4,4′-dichloro-indigo | Alkaline Phosphatase |
| d) | para-nitrophenyl phosphate | 4-nitrophenol + phosphoric acid | Alkaline Phosphatase |
| e) | 3,3′,5,5′- Tetramethylbenzidine (TMB) + H₂O₂ | 3,3′,5,5′-tetramethylbenzidine diamine | Horseradish peroxidase. |
| f) | 2,2′-azino-bis(3- ethylbenzothiazoline-6- sulphonic acid) (ABTS) + H₂O₂ | ABTS+ radical [2,2-azino-bis(ethylbenzene-thiazoline- 6-sulfonic acid)] | Horseradish peroxidase. |

Respective reactions are schematically illustrated in FIG. 11.

Enzymes were conjugated to antibodies or microspheres using various conjugation methods including:
a) Biotin-streptavidin: Enyzmes conjugated with streptavidin were linked to detection antibodies labeled with biotin;
b) EDC/NHS coupling: Proteins were covalently coupled to the surface of carboxylated microspheres (Estapor® Microsphères Latex 0.438 µm, Merck Millipore) following the manufacturer’s protocol (EMD Millipore Corp., “Microsphere Coupling - Two-step EDC/Sulfo NHS Covalent Coupling Procedure for Estapor® Carboxyl-modified Dyed Microspheres.” [Online]. Available: https://www.emdmillipore.com/Web-US-Site/en CA/-/USD/ShowDocument-Pronet?id=201507.096.);
c) Through activation of the carboxyl groups with water-soluble 1-ethyl-3-[3-dimethylaminopropyl] carbodiimide (EDC). The reaction forms an active intermediate (O-acylisourea) that reacts quickly with primary amines (nucleophile) to form a stable amide bond; and
d) Physical adsorption (the physical adsorption of enzymes and antibodies to cellulose microspheres was performed using the method described in the below section eFlow workflow).

Those skilled in the art will appreciate that other methods are possible.

FIGS. 6a to 6c show examples of pH and ORP signal generation by different enzymes at various concentrations in the presence of their respective substrates with an eELISA sensor chip as described.

FIG. 6a shows the pH signal generated by addition of 5 µl alkaline phosphatase (ranging from 10 ng/ml to 10 µg/ml in 0.02x phosphate buffered saline containing 100 mM NaCl) to 45 µl 0.02x phosphate buffered saline solution containing 100 mM NaCl and para-Nitrophenylphosphate (pNPP) (ranging from 15 mM to 100 mM pNPP). The reference well was filled with 1 M KCl solution.

FIG. 6b shows the ORP signal generated by adding 5 µl glucose oxidase (ranging from 1 ng/ml to 10 µg/ml in 0.02x phosphate buffered saline containing 100 mM NaCl) to 45 µl 0.02x phosphate buffered saline containing 100 mM NaCl, 200 mM β-D-(+) Glucose and 1% w/v or 0% v/w bovine serum albumin (BSA). The reference well was filled with 1 M KCl solution.

FIG. 6c shows the ORP signal generated by adding 5 µl horseradish peroxidase (ranging from 0.01 ng/ml to 10 ng/ml) in phosphate citrate buffer to 45 µl phosphate citrate buffer containing 1 mM ABTS (2,2′-Azinobis [3-ethylbenzothiazoline-6-sulfonic acid]-diammonium salt). The reference well was filled with 1 M KCl solution.

FIG. 7 shows an example for a conjugate consisting of 10 mg latex microspheres with immobilized Anti-VGEF antibodies (9 mg/g microspheres) and horseradish peroxidase (1 µl 1:60 dilution Poly-HRP Streptavidin, Thermo Scientific) in 1 ml 1x PBS buffer with 1% BSA. Using an eELISA sensor chip as described in the above eELISA sensor chip fabrication section, 5 µl of different conjugate dilutions (from 1:100 to 1:10,000,000 in PBS) are added to 45 µl substrate solution (2 mM ABTS in 100 mM phosphate citrate buffer pH 5.4 with 2 mM H₂O₂), and transferred to a test well. The reference well contains 100 mM phosphate citrate buffer pH 5.4. The ORP signal (potential difference between Pt electrode in a test well vs. Pt electrode in the reference well) is recorded over time. As seen in FIG. 7, a wide concentration range of conjugate can be quantified in real-time. This system allows to detect the presence of conjugate at a high dynamic range (six orders of magnitude within 15 min).

FIG. 8 shows the ORP signal generation of substrate conversion (3,3′,5,5′-Tetramethylbenzidine Liquid Substrate System for Membranes, Merck) using an antibody-enzyme conjugate (Goat Anti-Rabbit IgG Antibody, Fc, HRP conjugate, Sigma-Aldrich) immobilized on an LFA strip (nitrocellulose membrane, Merk Millipore) at 50 µg/ml in 1x PBS. The ORP voltage is measured between the gold electrodes of an eFlow sensor chip (as described in the above eFlow sensor chip fabrication section) in contact with the LFA test strip, wherein the “Test” curve indicates the electric potential difference between the test electrode and the reference electrode whereas the “Control” curve indicates the electric potential difference between the control electrode and the reference electrode (cf. FIG. 5b).

### eELISA Workflow

An ELISA kit for human VEGF R1/Flt-1 (DY321, R&D Systems, Minneapolis, USA) was used according to the general ELISA protocol (as provided with the DY321 kit from R&D Systems, in 2018). A 96-well high binding ELISA plate (Greiner Bio-one, Kremsmünster, Austria) was coated overnight with 100 µl primary antibodies, at a concentration of 2 µg/ml in 1x PBS. The next day, the plate was washed three times using a plate washer (BioTek’s ELx50) with 300 µl wash buffer (1x PBS + 0.05% Tween 20) per well. The plate was then blocked for one hour with 300 µl 1x PBS + 1% BSA per well. Afterwards the plate was washed again and 100 µl of the recombinant human VEGF R1 standard dilutions were added to each well in the following concentrations, and incubated for two hours: 8000 pg/ml, 4000 pg/ml, 2000 pg/ml, 1000 pg/ml, 500 pg/ml, 250 pg/ml and 125 pg/ml. After a further washing step, 100 µl of 500 ng/ml secondary antibodies were added per well and incubated for two hours. After subsequent washing, the enzyme Poly-HRP-SA (Poly-Horseradish peroxidase streptavidin, Thermo Scientific) was coupled to the antibody by adding 100 µl 10,000x dilution of Poly-HRP-SA to the well, incubated for 20 minutes, and washed three times using the above-mentioned washing procedure.

For eELISA measurements, 100 µl ABTS substrate solution (5 mg 2,2′-Azino-bis(3-ethylbenzothiazoline-6-sulfonic acid) diammonium salt (ABTS, Roche) dissolved in 5 ml 100 mM phosphate citrate buffer pH 5.4 with 2 mM H₂O₂) was added into the wells. End point measurements of the enzyme reaction were measured with the eELISA setup, by adding the converted substrate solution to the eELISA sensor chip and comparing it with the substrate solution baseline. All incubations were performed at room temperature.

FIG. 9a shows the eELISA readout. ORP sensor baseline was established using 50 µl ABTS substrate contained in the reference well. The solution in each test well was sequentially replaced by 50 µl converted substrate solutions of the ELISA plate. The difference in voltage corresponds to the change in oxidation-reduction potential and correlates with the VEGF R1 concentration. FIG. 9b shows an eELISA standard curve derived from FIG. 9a indicating the relationship between the electric potential difference and the concentration of the analyte, i.e. human VEGF R1.

### eFlow Workflow

Fabrication of lateral flow assay test strips: Immobilization of test and control lines was done by lining capture antibodies on a conventional backed nitrocellulose membrane (Hi-Flow Plus 120 Membrane Cards 60 mm × 301 mm, HF120MC100 Merck Millipore) using a BioJet liner (BioDot Inc.), and dried at room temperature for 2 hours. Test line: Mouse anti-h CRP 6404 SP-6 (Oy Medix Biochemica Ab, Espoo, Finland), 1 mg/ml in 1x PBS buffer pH 7.4. Control line: AffiniPure Goat Anti-Mouse IgG F(ab′)2 Fragment (Jackson ImmunoResearch), 1 mg/ml in 1x PBS buffer pH 7.4. An absorbent pad was fixed at the end of the strip using the adhesive on the membrane backing.

Microsphere conjugates containing detection antibody and enzyme were prepared as follows. 30 µl of 1% beads (NanoAct cellulose beads, Asahi Kasei) were sonicated for 10 s and mixed with 270 µl conjugate buffer (10 mM Tris, pH 7), 3.2 µl detection antibody (anti-h CRP 6405 4.7 mg/ml in 1x PBS), and 44 µl HRP (peroxidase from horseradish type VI 1 mg/ml in 1x PBS, Sigma Aldrich), vortexed and kept at 37° C. for 120 min. Afterwards, 3.6 ml of blocking buffer (1% Casein, 100 mM Borate pH 8.5) was added and the mix was kept at 37° C. for 60 min. After blocking, the beads were washed by centrifuge at 13,000 g for 20 min followed by removal of the supernatant and addition of washing buffer (50 mM Borate pH 10.0), sonication for 10 sec, centrifuge at 13,000 g for 20 min, removal of the supernatant, and addition of drying buffer (0.2% Casein, 15% Sucrose, 33 mM Borate pH 9.2).

The testing procedure involved mixing 5 µl of conjugates with 45 µl of CRP standard solutions (recombinant C-reactive protein in running buffer: PBS, 10% Sucrose, 0.1% BSA) at 10,000 pg/ml, 2000 pg/ml, 400 pg/ml, 80 pg/ml and 0 pg/ml in microwells of a 96-well ELISA plate. The test strips were dipped vertically into the wells, then letting the membrane and absorbent pad soak the solution for 20 min. A colorimetric signal at the control line indicates the correct execution of the test (see FIG. 10a, which shows the lateral flow assay test strips after running 2000, 400, 80, 0 pg/ml C-reactive protein mixed with conjugates).

After running the LFA, the absorbent pad was removed from the test strips. The test strips were then placed in microwells containing 20 µl of substrate solution (5 mg 2,2′-Azino-bis(3-ethylbenzothiazoline-6-sulfonic acid) diammonium salt (ABTS) dissolved in 1 ml 100 mM phosphate citrate buffer pH 5.4 with 10 mM H₂O₂). After 5 min, the substrate was soaked by the nitrocellulose membrane, and the test strips where placed on the electrical measurement setup in contact with the eFlow sensor chips. The reference electrode (platinum electrode, defining the reference potential of the nitrocellulose membrane outside of the immobilization areas) was set to ground and the voltage change at each platinum ORP electrode was recorded with respect to the reference electrode.

Referring to FIG. 10b, the result of the eFlow measurements shows the test line ORP signal (voltage between a Pt electrode at test line and a Pt electrode outside of immobilization areas) for samples containing different C-reactive protein concentrations. The signal corresponds to the enzymatic conversion of ABTS and H₂O₂ correlating to the conjugate concentrations (depending on analyte concentration).

While the invention has been illustrated and described in detail in the drawings and foregoing description, such illustration and description are to be considered illustrative or exemplary and non-restrictive; the invention is thus not limited to the disclosed embodiments. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality and may mean “at least one”.

The invention may be defined, for example, in accordance with the following items:

1. An immunoassay analyzer for detecting at least one analyte in a liquid sample, the immunoassay analyzer comprising:
a transport matrix for transporting the liquid sample to at least one immobilization area along the transport matrix;
at least one first capture reagent immobilized in the at least one immobilization area, the first capture reagent capable of binding with and thereby capturing the at least one analyte; and
at least one amperometric and/or potentiometric sensor element comprising at least one first working electrode and at least one reference electrode, wherein the first working electrode is configured to contact with the at least one immobilization area and the reference electrode is configured to contact with a portion of the transport matrix other than the at least one immobilization area.

2. The immunoassay analyzer of item 1, wherein the first capture reagent is immobilized in a first immobilization area and capable of binding with and thereby capturing a first analyte in the liquid sample,
the immunoassay analyzer further comprising:
a second capture reagent immobilized in a second immobilization area, the second capture reagent capable of binding with and thereby capturing a second analyte in the liquid sample, the second immobilization area being downstream of the first immobilization area or the second immobilization area and the first immobilization area being located, respectively, in isolated flow paths,
wherein the sensor element further comprises a second working electrode configured to contact with the second immobilization area.

3. The immunoassay analyzer of item 1 or 2, further comprising a read-out circuit configured to determine an electric potential difference between the first working electrode and the reference electrode and/or a change in an electric current flowing through the first working electrode and the reference electrode, preferably the read-out circuit being configured to further determine an electric potential difference between the second working electrode and the reference electrode and/or a change in an electric current flowing through the second working electrode and the reference electrode.

4. The immunoassay analyzer of any of the preceding items, wherein the read-out circuit is configured to determine a respective amount of the at least one analyte based on the respective electric potential difference and/or the respective change in the electric current.

5. The immunoassay analyzer of any of the preceding items, where the sensor element further comprises a backing and/or support structure on which the working electrode and the reference electrode are provided.

6. The immunoassay analyzer of any of the preceding items, wherein the first and/or second working electrode is an ion-selective electrode (ISE) such as a pH electrode or an oxidation-reduction potential (ORP) electrode.

7. The immunoassay analyzer of any of the preceding items, wherein the first and/or second working electrode comprises a noble metal such as platinum, gold or silver or a layer of metal oxides such as Al₂O₃, HfO₂ or Ta₂O₅, preferably the working electrode comprising a layer of titanium superimposed by a layer of Ta₂O₅.

8. The immunoassay analyzer of any of the preceding items, wherein the reference electrode comprises a metal such as Ti, Ag, Au, Pt or a combination thereof or a metal salt such as Ag/AgCl, preferably the reference electrode does not comprise a metal oxide such as Al₂O₃, HfO₂ or Ta₂O₅.

9. The immunoassay analyzer of any of the preceding items, wherein the sensor element is adhered to the transport matrix or brought into contact with the transport matrix by a mechanical pressure in a manner that the working electrode is in contact with the immobilization area and the reference electrode is in contact with the portion of the transport matrix other than the at least one immobilization area.

10. The immunoassay analyzer of any of the preceding items, wherein the capture reagent comprises an antibody, an engineered protein, DNA, RNA or an aptamer.

11. The immunoassay analyzer of any of the preceding items, wherein the transport matrix comprises a microporous membrane strip, preferably the membrane strip comprising a polymer such as nitrocellulose or an inorganic material such as glass fibers.

12. The immunoassay analyzer of the preceding item, further comprising a sample pad at a first end of the membrane strip for introducing the liquid sample to the transport matrix.

13. The immunoassay analyzer of item 11 or 12, further comprising a reagent pad at the first end of the membrane strip and in fluid communication with the at least one immobilization area.

14. The immunoassay analyzer of any of items 1 to 10, wherein the transport matrix comprises a liquid flow cell.

15. The immunoassay analyzer of any of the preceding items, further comprising a conjugate, the conjugate comprising a binding molecule specific to the at least one analyte or to the at least one capture reagent, the binding molecule preferably being an antibody, an engineered protein, DNA, RNA or an aptamer.

16. The immunoassay analyzer of the preceding item, wherein the conjugate further comprises: an enzyme, and/or a redox-active molecule such as ferrocyanide ions, methylene blue or resazurin.

17. The immunoassay analyzer of item 16, wherein the enzyme comprises glucose oxidase, glucose-6-phosphate dehydrogenase, urease, alkaline phosphatase and/or horseradish peroxidase.

18. The immunoassay analyzer of any of items 15 to 17, wherein the conjugate further comprises microspheres or colloidal metal.

19. The immunoassay analyzer of any of items 15 to 18, further comprising a substrate convertible into a product via a reaction with the conjugate, preferably via an enzymatic reaction.

20. The immunoassay analyzer of the preceding item, wherein the substrate comprises D-glucose, glucose 6-phosphate/NADP⁺; nitro-blue tetrazolium/5-bromo-4-chloro-3′-indolyl phosphate (NBT/BCIP); para-nitrophenyl phosphate (p-NPP); 3,3′,5,5′-Tetramethylbenzidine (TMB)/H₂O₂ and/or 2,2′-azino-bis(3-ethylbenzothiazoline-6-sulphonic acid) (ABTS)/H₂O₂.

21. The immunoassay analyzer of item 19 or 20, wherein
the substrate comprises a substance capable of lowering the diffusibility of the product, the substance preferably comprising saccharides or gels, and/or
the reaction produces a precipitate, preferably wherein an enzymatic reaction between the substrate and the conjugate produces the precipitate.

22. An immunoassay kit, comprising
the immunoassay analyzer of any of items 1 to 14,
a conjugate comprising a binding molecule specific to the at least one analyte, and
a substrate convertible into a product via a reaction with the conjugate.

23. An immunoassay analyzer for detecting an analyte in a liquid sample, the immunoassay analyzer comprising:
at least one microwell plate comprising at least one reference well and at least one test well, the reference well connected to each test well via a microfluidic channel;
at least one reference electrode in the reference well; and
a first working electrode and/or a second working electrode in each test well, the first working electrode and the second working electrode being separated from each other.

24. The immunoassay analyzer of item 23, further comprising a read-out circuit for determining a first electric potential difference between the first working electrode and the reference electrode and/or a second electric potential difference between the second working electrode and the reference electrode.

25. The immunoassay analyzer of item 23 or 24, wherein the first working electrode comprises a layer of titanium superimposed by a layer of Ta₂O₅.

26. The immunoassay analyzer of any of items 23 to 25, wherein the second working electrode comprises a layer of titanium superposed by a layer of platinum.

27. The immunoassay analyzer of any of items 23 to 26, wherein the reference electrode comprises a layer of titanium superposed by a layer of platinum.

28. The immunoassay analyzer of any of items 23 to 27, wherein
the first working electrode is a pH electrode, and/or
the second working electrode is an ORP electrode.

29. The immunoassay analyzer of any of items 23 to 28, wherein the reference well is connected to each test well via a salt bridge in the microfluidic channel, the salt bridge preferably comprising an electrolyte and/or a gel.

30. The immunoassay analyzer of any of items 23 to 29, wherein the analyte is immobilized in one or more of the test wells.

31. The immunoassay analyzer of any of items 23 to 29, further comprising a capture reagent immobilized in each test well, the capture reagent capable of binding with and thereby capturing the analyte.

32. The immunoassay analyzer of any items 23 to 31, further comprising a conjugate, the conjugate comprising a binding molecule specific to the at least one analyte or to the at least one capture reagent, the binding molecule preferably being an antibody, an engineered protein, DNA, RNA or an aptamer.

33. The immunoassay analyzer of the preceding item, wherein the conjugate further comprises:
an enzyme, and/or
a redox-active molecule such as ferrocyanide ions, methylene blue or resazurin.

34. The immunoassay analyzer of item 33, wherein the enzyme comprises glucose oxidase, glucose-6-phosphate dehydrogenase, urease, alkaline phosphatase and/or horseradish peroxidase.

35. The immunoassay analyzer of any of items 33 to 34, wherein the conjugate further comprises microspheres or colloidal metal.

36. The immunoassay analyzer of any of items 33 to 35, further comprising a substrate convertible into a product via a reaction with the conjugate, preferably via an enzymatic reaction.

37. The immunoassay analyzer of the preceding item, wherein the substrate comprises D-glucose, glucose 6-phosphate/NADP⁺; nitro-blue tetrazolium/5-bromo-4-chloro-3′-indolyl phosphate (NBT/BCIP); para-nitrophenyl phosphate (p-NPP); 3,3′,5,5′-Tetramethylbenzidine (TMB)/H₂O₂ and/or 2,2′-azino-bis(3-ethylbenzothiazoline-6-sulphonic acid) (ABTS)/H₂O₂.

38. An immunoassay kit, comprising the immunoassay analyzer of any of items 23 to 37, a conjugate comprising a binding molecule specific to the analyte, and a substrate convertible into a product via a reaction with the conjugate.

39. A method for detecting at least one analyte in a liquid sample, the method comprising:
a) binding the at least one analyte in the liquid sample with a capture reagent;
b) binding a conjugate to the at least one analyte or the capture reagent;
c) providing a substrate and converting the substrate into a product via a reaction with the conjugate, wherein the product is provided in a test area; and
d) detecting an electric potential difference and/or a change in an electric current between at least one working electrode in the test area and at least one reference electrode in a reference area which is different from the test area.

40. The method of the preceding item, further comprising:
e) determining an amount of the at least one analyte based on the electric potential difference and/or the change in electric current.

41. The method of item 39 or 40, wherein step a) comprises introducing the liquid sample to a transport matrix for transporting the liquid sample, the transport matrix comprising the test area, wherein the capture reagent is immobilized in the test area.

42. The method of the preceding item, wherein step b) comprises providing the conjugate in the test area of the transport matrix.

43. The method of the preceding item, wherein providing the conjugate comprises:
providing the conjugate in the test area after the at least one analyte has bound to the capture reagent, or
mixing the conjugate with the liquid sample before the liquid sample is introduced to the transport matrix.

44. The method of the preceding item, wherein mixing the conjugate with the liquid sample comprises:
mixing the liquid sample with a buffer solution containing the conjugate before the liquid sample is introduced to the transport matrix, or
providing the liquid sample in a reagent pad in contact and/or fluidly coupled with the transport matrix, wherein the reagent pad contains the conjugate.

45. The method of any of items 41 to 44, wherein step c) comprises providing the substrate in the test area of the transport matrix.

46. The method of the preceding item, wherein providing the substrate comprises:
providing the liquid sample in a reagent pad in contact and/or fluidly coupled with the transport matrix, wherein the reagent pad contains the substrate, or
providing the substrate in the test area along with the liquid sample, or
providing a buffer solution containing the substrate in the test area after the liquid sample has been transported to the test area and the conjugate has been bound to the at least one analyte or the capture reagent.

47. The method of any of items 41 to 46, wherein step d) comprises:
providing a sensor element comprising the working electrode and the reference electrode; and
contacting the sensor element with the transport matrix in a manner that the working electrode is in contact with the test area and the reference electrode is in contact with the reference area, preferably wherein the reference area is a portion of the transport matrix other than the test area and/or a portion of the transport matrix in which the capture reagent is not immobilized.

48. The method of item 39 or 40, further comprising:
providing at least one microwell plate comprising at least one reference well and at least one test well defining the test area, the reference electrode being provided in the reference well and the working electrode being provided in the test well, the reference well being connected to the test well via at least one microfluidic channel, wherein step d) is carried out using the microwell plate.

49. The method of item 48, further comprising

immobilizing the analyte in the test well.

50. The method of item 48, further comprising
immobilizing the capture reagent in the test well,
preferably wherein step a) comprises providing the liquid sample in the test well and/or in the reference well.

51. The method of item 48, 49 or 50, wherein step b) comprises providing the conjugate in the test well and/or in the reference well.

52. The method of any of items 48 to 51, wherein step c) comprises providing the substrate in the test well and/or in the reference well.

53. The method of any of items 39 to 52, wherein the at least one analyte comprises one of ions, molecules, proteins, pathogens, DNA and/or RNA.

1. An immunoassay analyzer for detecting at least one analyte in a liquid sample, the immunoassay analyzer comprising:
a transport matrix for transporting the liquid sample to at least one immobilization area along the transport matrix;
at least one first capture reagent immobilized in the at least one immobilization area, the first capture reagent capable of binding with and thereby capturing the at least one analyte; and
at least one amperometric or potentiometric sensor element comprising at least one first working electrode and at least one reference electrode, wherein the first working electrode is configured to contact with the at least one immobilization area and the reference electrode is configured to contact with a portion of the transport matrix other than the at least one immobilization area.

2. The immunoassay analyzer of claim 1, wherein the first capture reagent is immobilized in a first immobilization area and capable of binding with and thereby capturing a first analyte in the liquid sample,
the immunoassay analyzer further comprising:
a second capture reagent immobilized in a second immobilization area, the second capture reagent capable of binding with and thereby capturing a second analyte in the liquid sample, the second immobilization area being downstream of the first immobilization area or the second immobilization area and the first immobilization area being located, respectively, in isolated flow paths,
wherein the sensor element further comprises a second working electrode configured to contact with the second immobilization area.

3. The immunoassay analyzer of claim 1, further comprising a read-out circuit configured to determine an electric potential difference between the first working electrode and the reference electrode or a change in an electric current flowing through the first working electrode and the reference electrode.

4. The immunoassay analyzer of claim 1, wherein the first or second working electrode is an ion-selective electrode (ISE).

5. The immunoassay analyzer of claim 1, wherein the reference electrode comprises platinum or gold.

6. The immunoassay analyzer of claim 1, further comprising a conjugate, the conjugate comprising a binding molecule specific to the at least one analyte or to the at least one capture reagent.

7. The immunoassay analyzer of claim 6, further comprising a substrate convertible into a product via a reaction with the conjugate.

8. The immunoassay analyzer of claim 7, wherein
the substrate comprises a substance capable of lowering the diffusibility of the product, or
the reaction produces a precipitate.

9-17. (canceled)

18. An immunoassay kit, comprising the immunoassay analyzer of claims 1, a conjugate comprising a binding molecule specific to the analyte, and a substrate convertible into a product via a reaction with the conjugate.

19. A method for detecting at least one analyte in a liquid sample, the method comprising:
a) binding the at least one analyte in the liquid sample with a capture reagent;
b) binding a conjugate to the at least one analyte or the capture reagent;
c) providing a substrate and converting the substrate into a product via a reaction with the conjugate, wherein the product is provided in a test area; and
d) detecting an electric potential difference or a change in an electric current between at least one working electrode in the test area and at least one reference electrode in a reference area which is different from the test area.

20. The method of claim 19, further comprising:
e) determining an amount of the at least one analyte based on the electric potential difference or the change in electric current.

21. The method of claim 19, wherein step a) comprises introducing the liquid sample to a transport matrix for transporting the liquid sample, the transport matrix comprising the test area, wherein the capture reagent is immobilized in the test area.

22. The method of claim 21, wherein step b) comprises providing the conjugate in the test area of the transport matrix.

23. The method of claim 21, wherein step c) comprises providing the substrate in the test area of the transport matrix.

24. The method of claim 21, wherein step d) comprises:
providing a sensor element comprising the working electrode and the reference electrode; and
contacting the sensor element with the transport matrix in a manner that the working electrode is in contact with the test area and the reference electrode is in contact with the reference area.

25-28. (canceled)

29. The method of claim 24, wherein the reference area is a portion of the transport matrix other than the test area or a portion of the transport matrix in which the capture reagent is not immobilized.

30. The immunoassay analyzer of claim 4, wherein the first or second working electrode is a pH electrode or an oxidation-reduction potential (ORP) electrode.

31. The immunoassay analyzer of claim 3, wherein the read-out circuit is configured to further determine an electric potential difference between the second working electrode and the reference electrode or a change in an electric current flowing through the second working electrode and the reference electrode.

32. The immunoassay analyzer of claim 31, wherein the read-out circuit is configured to determine a respective amount of the at least one analyte based on the respective electric potential difference or the respective change in the electric current.

33. The immunoassay analyzer of claim 6, wherein the binding molecule is an antibody, an engineered protein, DNA, RNA or an aptamer, and wherein the conjugate further comprises one of an enzyme and a redox-active molecule.

34. The immunoassay analyzer of claim 7, wherein the substrate is convertible into a product via an enzymatic reaction with the conjugate.
